(19) United States
(12) Patent Application Publication
(10) Pub. No.: US 2023/0285106 A1
(43) Pub. Date: Sep. 14, 2023

(54) PROTECTIVE NASAL MASK DEVICE

(71) Applicant: Waterford Institute of Technology, Waterford (IE)

(72) Inventors: Ramesh RAGHAVENDRA, Waterford (IE); Eoghan O'DONAGHUE, Waterford (IE); Brendan PHELAN, Waterford (IE); Colin REIDY, Waterford (IE); Nataraj Bangalore MAHESH, Waterford (IE); David SMYTH, Waterford (IE)

(21) Appl. No.: 17/927,495

(22) PCT Filed: May 31, 2021
(86) PCT No.: PCT/EP2021/064587

§ 371 Date: Nov. 23, 2022

(30) Foreign Application Priority Data
May 29, 2020 (EP) 20177557.4

(51) Int. Cl. A61B 90/00; A61B 17/24; A61G 10/00

(52) U.S. Cl. CPC A61B 90/05; A61B 17/24; A61G 10/005; A61B 2017/345

(57) ABSTRACT

The present invention relates to a protective nasal mask device which hermetically seals the nasal region of a user during an ENT procedure. The device facilitates access to the user's nose and throat using a plurality of surgical tools. The primary advantage of the present invention is to provide a nasal mask device which protects medical professionals from disease transmission during an ENT procedure. Additional advantages include a nasal mask device which enables usage of a plurality of surgical tools during an ENT procedure, a device which at the same time, prevents exposure of biohazardous waste to medical professionals. The device hermetically seals the nasal region of a patient during an ENT procedure.

### FIELD

The present invention relates to a protective nasal mask device to protect a surgeon or clinician during a procedure. In particular the invention relates to a nasal mask device enabling protection to medical professionals against disease transmission during Ear, Nose and Throat (ENT) Procedures.

### BACKGROUND TO THE INVENTION

The coronavirus disease 2019 (COVID-19) is probably the gravest global health crisis of our time and one of the greatest challenges' humanity has faced at least since World War-2. Since its emergence in December 2019, COVID-19 has spread worldwide and has been classified as a pandemic by the World Health Organisation. Medical professionals and health care workers who are at the front line of the COVID-19 outbreak response are exposed to the greatest risk of contracting the disease. To add to this, countries severely affected by COVID-19, have also been experiencing a dire shortage of Personal Protective Equipment (PPE), which further accentuates the risk of medical professionals getting infected.

Global experience with COVID-19 suggest that it poses a significant risk of infectious transmission to skull base surgeons, due to high nasal viral exposure and the unknown potential for aerosol generation during the endonasal procedure. In this pandemic, medical professionals dealing with conditions pertaining to the respiratory system are particularly at risk and those undertaking ENT procedures would have greater risk of exposure to the virus. A typical ENT procedure is the inspection of the nasal cavity of a patient using Naso-endoscopy which is a procedure where an endoscopic camera with a flexible tip is introduced into the nasal cavity through the nostrils. This can allow inspection of not only the nasal cavity itself but also the patient's throat and even vocal cords allowing many different conditions to be investigated and diagnosed. Further, a patient coughing or sneezing releases aerosolised particles that can sit in the air for up to three hours and hence becomes persistent in the environment. This maximises the chances of disease transmission not only to medical practitioners but also other patients. Medical practitioners do not have the option of disregarding these often critical and essential medical procedures and therefore, a means for safely performing such diagnostic and surgical procedures with minimum risk to the clinician is required. It is a safe assumption that the current pandemic would most likely entirely change the way medical care is delivered in the future. Therefore, finding a solution to the above-mentioned problem is essential.

Nasal mask devices used in the treatment of respiratory conditions and sleep disorders, for example as disclosed in U.S. Pat. No. 6,019,101 are well known in the art. However, such nasal masks are designed to provide supplementary respiratory support to the user.

US Patent Publication Number US 2012/285461 discloses a mask for patient ventilation. One embodiment shows an interchangeable insert with a self-sealing access port designed so that the mask does not need to be removed whilst allowing access to the patient. The mask is designed primarily for ventilation only. None of the devices known in the art are designed to ensure protection to the clinician from pathogens during an ENT procedure/examination.

For example, the ‘interchangeable faceplate’ of US 2012/285461, which is primarily designed at ventilation, is positioned too close to the nose of a patient to actually be useful for the full range of ENT surgical procedures. The interchangeable faceplate itself is an unnecessary complication and can be prone to failure during surgery. The interchangeable faceplate does not protect the surgeon or clinician. Moreover the facemask as intended is not designed to cope with waste material such as blood and infected tissue material biohazardous waste being removed from the patient during a surgery.

There is therefore an unfulfilled and unresolved need for a device which provides protection to medical professionals against disease transmission during Ear, Nose and Throat (ENT) procedures, while facilitating such procedures at the same time.

### SUMMARY OF THE INVENTION

The present invention relates to a protective nasal mask device which hermetically seals the nasal region of a user during an ENT procedure, as set out in the appended claims. The device as per the present invention further facilitates access to the user's nose and throat using a plurality of surgical tools.

The primary advantage of the present invention is to provide a protective nasal mask device which protects medical professionals from disease transmission during an ENT procedure. Additional advantages include a nasal mask device which enables usage of a plurality of surgical tools during an ENT procedure, a device which at the same time, prevents exposure of biohazardous waste to medical professionals. The device which hermetically seals the nasal region of a patient during an ENT procedure.

In one embodiment of the present invention, a nasal mask device is provided which comprises a sealing means, a device body and a plurality of tool passing channels. Sealing refers to hermetically sealing the device to the nasal and mouth region of the user. The top edge of the sealing means is coupled to the device body. The device body has a flexible elastomeric member formed on its bottom edge. The plurality of tool passing channels run longitudinally and are angled inwardly towards each other. The bottom end of each tool passing channel has an access port formed on it and the top end opens to a nostril of the user. The access port enables introduction of surgical tools to a nostril of a user during an ENT procedure such as a Naso-endoscopy or clinical assessment procedures. Each access port allows insertion of only one surgical tool at a time.

In one embodiment one of the ports is adapted to allow the user to breathe normally while the mask is in place.

In one embodiment the nasal mask device is dimensioned to fit over the nose and mouth of a patient.

Therefore, using the plurality of access ports formed in the plurality of tool passing channels, multiple surgical tools can be introduced at the same time during an ENT procedure. The tool passing channels are mounted on the flexible elastomeric member which enables independent movement of each tool passing channel. Each of the access ports are adapted to seal itself once a surgical tool inserted through it is removed thereby ensuring non permeation of any disease-causing pathogens outside the nasal mask device. At least one pouch is formed adjacent to each access port and inside the tool passing channel. Each pouch opens towards the top end of the tool passing channel which allows it to collect any biohazardous waste generated during the procedure.

In another embodiment there is provided a nasal mask device comprising: a sealing means adapted to hermetically seal the device to the nasal region of a user, the sealing means having a bottom edge and a top edge disposed opposite to the bottom edge; a device body coupled to the top edge of the sealing means, the device body having a flexible member integrated to its bottom edge; and at least one longitudinally running tool passing channel mounted on the flexible member and angled inwardly towards each other, the tool passing channel having a bottom end with an access port formed on it and a top end kept open, the access port having at least one pouch formed adjacent to it and inside the tool passing channel such that the pouch opens towards the top end of the tool passing channel, wherein the flexible member enables independent movement of the tool passing channel and wherein the access port is adapted to seal itself when a surgical tool inserted through it is removed. It will be appreciated that the nasal mask device of the invention can incorporate any number of ports, depending on the application required.

In one embodiment at least one of the tool passing channels is adapted to provide an air passageway to enable a patient to breathe.

In one embodiment at least one access port comprises at least one pouch formed adjacent to it and inside the tool passing channel such that the pouch opens towards the top end of the tool passing channel.

In one embodiment the pouch is designed to securely store hazardous material from a user or patient during a procedure.

In one embodiment at least one pouch is adapted as a store area for storage of materials required during a procedure, whilst maintaining the seal with the user or patient.

As per another preferred embodiment of the present invention there is a method provided for performing an ENT procedure, the method comprises of the steps of hermetically sealing the nasal region of a patient using a nasal mask device and further introducing a plurality of surgical tools through the nasal mask device. The nasal region does not include the mouth of the patient. The nasal mask device comprises a plurality of longitudinally running tool passing channels. Each tool passing channel has an access port formed on its bottom end and its top end opens to a nostril of the user. Each access port allows introduction of surgical tools during the procedure. Only one surgical tool can be inserted through each access port at a time. Further, each access port has at least one pouch formed adjacent to it and inside the tool passing channel such that each pouch opens towards the top end of each of the tool passing channels. The method as per said preferred embodiment further comprises the steps of collecting biohazardous waste generated during the procedure using the plurality of pouches formed in the tool passing channel, and sealing each access port once the surgical tool inserted through it is removed.

In another embodiment there is provided a protective nasal mask device comprising a sealing means adapted to hermetically seal the device to the nasal region of a user, the sealing means having a bottom edge and a top edge disposed opposite to the bottom edge; a device body coupled to the top edge of the sealing means, the device body having a flexible member integrated to its bottom edge; and at least one longitudinally running tool passing channel mounted on the flexible member and angled inwardly towards each other, the tool passing channel having a bottom end with an access port formed on it and a top end kept open, wherein the flexible member enables independent movement of the tool passing channel and wherein the access port is adapted to seal itself when a surgical tool inserted through it is removed.

In one embodiment the device comprises at least one pouch formed adjacent to it and inside the tool passing channel such that the pouch opens towards the top end of the tool passing channel.

In one embodiment the device and tool passing channel comprises an elongated flexible chamber and coupled at one end with a flexible sealing means.

In one embodiment the access port is positioned at the opposite end of the elongated flexible camber.

In one embodiment a second access port is positioned near the flexible sealing means and configured to provide an air passageway to enable a patient to breathe.

In one embodiment the elongated flexible chamber comprises a transparent material.

The invention provides a method for performing an ENT procedure using a nasal mask device which prevents exposure to disease causing pathogens and other biohazardous waste.

The present invention therefore ensures that a clinician is not exposed to any disease-causing pathogens or biohazardous waste during an ENT procedure. It also facilitates usage of a plurality of surgical tools at the same time during the procedure.

Further, since the nasal mask device is hermetically sealed to the user the aerosolised particles released by the patient are confined to the device. The present invention hence provides a simple and low-cost solution which ensures safety of medical practitioners especially clinicians involved in ENT practice and allows them to continue their lifesaving work. Further, the design of the present invention ensures that it can be mass produced with ease.

The protective nasal mask device as per the present invention has wide industrial application and is expected to be of demand across the world. To illustrate the market potential of the device disclosed by the present invention—sinusitis is a very common health condition affecting 31 million Americans each year, 30% of the United States population have sinusitis at some point in their lives. An average American adult has three to four upper respiratory infections a year; 1% of these infections are complicated by sinusitis, accounting for 16 million visits to the doctor each year.

The nasal mask device disclosed in the present invention hence provides a robust and economical solution to problems identified in the art.

### BRIEF DESCRIPTION OF THE DRAWINGS

The invention will be more clearly understood from the following description of an embodiment thereof, given by way of example only, with reference to the accompanying drawings, in which:—

FIG. 1 illustrates a side view of a preferred embodiment of the present invention.

FIG. 2 illustrates a front view of a preferred embodiment of the present invention.

FIG. 3 illustrates a perspective view of a plurality of tool passing channels as per a preferred embodiment of the present invention.

FIG. 4 illustrates a cross sectional view of a preferred embodiment of the present invention.

FIG. 5 illustrates a further embodiment of the invention showing four tool passing channels.

FIG. 6 illustrates a prior art implementation of a clinician performing an ENT procedure on a patient.

FIG. 7 illustrates a side view of another embodiment of the present invention.

FIG. 8 illustrates a front view of the embodiment shown in FIG. 7.

FIG. 9 illustrates a 3D perspective view of the embodiment shown in FIGS. 7 and 8.

### DETAILED DESCRIPTION OF THE DRAWINGS

The present invention relates to a protective nasal mask device, and more particularly to a nasal mask device enabling protection to medical professionals against disease transmission during Ear, Nose and Throat (ENT) Procedures.

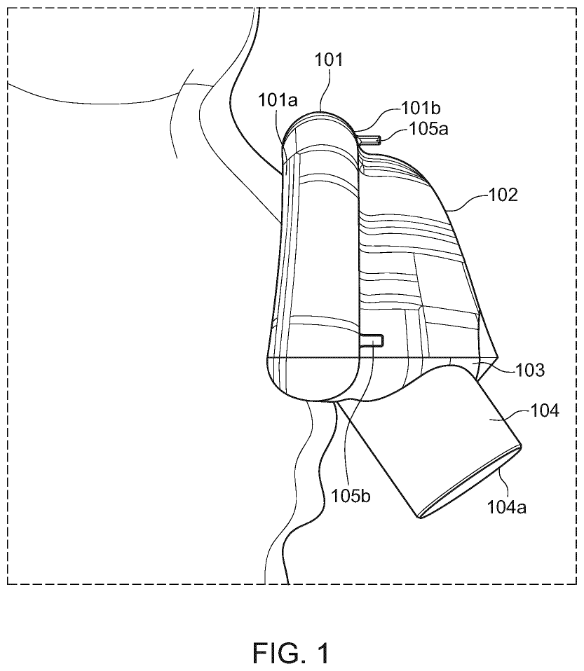

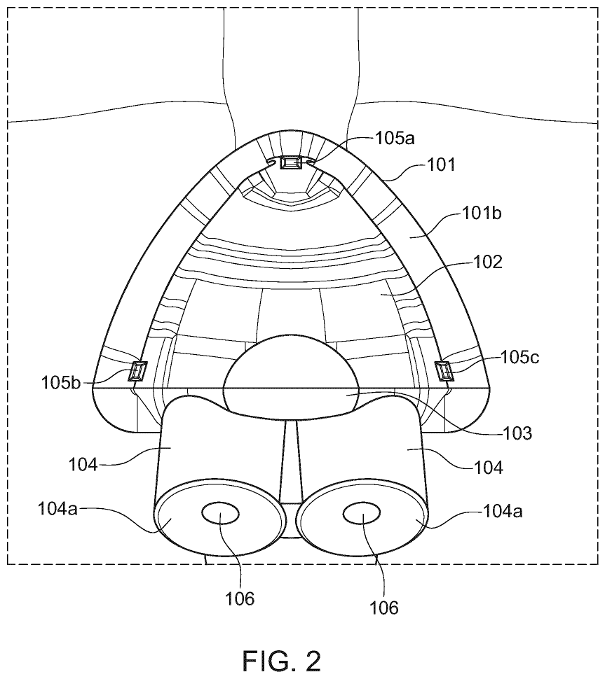

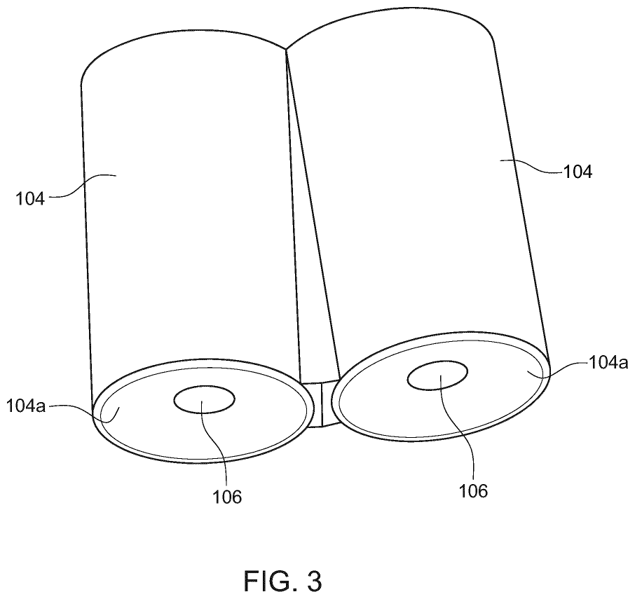

Referring to FIG. 1, FIG. 2 and FIG. 3, the nasal mask device as per a preferred embodiment of the present invention comprises a sealing means 101, a device body 102 and a plurality of tool passing channels 104.

The sealing means 101 has a bottom edge 101a and a top edge 101b. The bottom edge 101a hermetically seals the device to the nasal region of a user. The nasal region does not include the mouth of the user to enable the user to be intubated during the procedure. Hermitically sealing the nasal region ensures that aerosolised particles released by the user are not allowed to escape outside the nasal mask device. The sealing means 101 as per a preferred embodiment of the present invention comprises of elastomeric material. The top edge 101b of the sealing means 101 is coupled to the device body 102.

The device body has a flexible member 103 integrated to its bottom edge and has mounted on it a plurality of fixing points (105a, 105b, 105c) capable of securing the device to the nasal region of the user. The fixing points provides means for fixing/securing the device to the nasal region of the user. The device body 102 as per a preferred embodiment of the present invention comprises of transparent plastic and the flexible member 103 is comprised of elastomeric material.

A plurality of tool passing channels 104 are mounted on the flexible member 103. The plurality of tool passing channels 104 run longitudinally and are angled inwardly towards each other. Each tool passing channel 104 has a hollow interior and a bottom end 104a with an access port 106 formed on it. The top end 104b of each tool passing channel 104 opens to one of the nostrils of the user. Each access port 106 enables introduction of a surgical tool to access the nose and throat of the user. All the access ports formed in the plurality of tool passing channels 104 in combination provides access to a plurality of surgical tools simultaneously during an ENT procedure. However, at a time, only one surgical tool can be inserted through each access port 106.

As per one embodiment of the present invention, the diameter of each access port is six millimetres or in other words, the diameter of a surgical tool that can be used is six millimetres. At least one pouch is formed adjacent to each access port 106. The pouches are formed inside the tool passing channel 104 and open towards the top end 104b of each tool passing channel 104. Each access port 106 is adapted to seal itself once a surgical tool is removed from it. This ensures non permeation of any disease-causing pathogens from the nasal mask device.

Further, the pouch or pouches formed adjacent to each access port 106 allows collection of bloody patties, extracted tissue, material swabs, and other biohazardous waste without having to remove them out of the nasal mask device. The one or more pouches (not shown) can be formed to be part of the flexible membrane and positioned to allow ease of access by a clinician or surgeon. In particular the righthand side pouch can be positioned to allow ease of access from the left hand tool and the left hand pouch will be positioned to allow ease of access from the right hand tool. Each tool port can have at least one or more associated storage pouches to allow ease of use for tools with a variety of tip angles. Additionally, the pouches can be used as a storage area for materials likely to necessary during a procedure: i.e. typically necessary materials could be placed into the storage pouches before application of the mask to a patient.

The present invention hence provides an encapsulated environment around the user's nasal region while allowing access to surgical tools at the same time.

The flexible member 103 enables independent movement of each tool passing channel 104. This ensures that a plurality of surgical tools can be introduced through the tool passing channels 104 at different angles simultaneously. Both the flexible member 103 and tool passing channels 104 comprises of material that is durable enough to prevent damages/tears due to piercing with the surgical tools during the procedure. Further, as aforementioned, the plurality of tool passing channels 104 are angled inwardly towards each other and the top end 104b of each tool passing channel opens to a nostril of the user. This enables surgical tools to be directed to the nostrils of the user. The angle at which the tool passing channels 104 are positioned with respect to each other is preferably selected such that the outer ends 104a are spaced apart whereas the other ends angle towards each other, as shown in FIG. 3. This enables better access for the surgical tools and provides greater flexibility for the clinician or surgeon when maneuvering a surgical tool. A preferred embodiment of the present invention comprises of four tool passing channels.

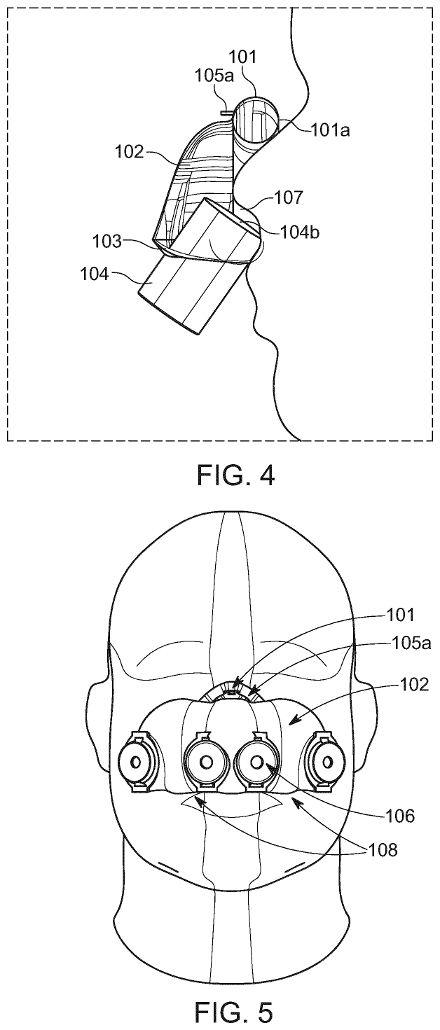

Referring to FIG. 4, the top end 104b of the tool passing channel 104 can be seen to be opening to the nostril 107 of the user. The plurality of tool passing channels 104 can also be mounted to the flexible member 103. As aforementioned, the flexible member 103 allows independent movement of each tool passing channel 104 and allows variety of angles of approach for each surgical tool.

As per another preferred embodiment of the present invention, there is provided a method for performing an ENT procedure. The method comprises the steps of hermetically sealing the nasal region of a patient using a nasal mask device, introducing at least one surgical tool through a plurality of access ports 106 formed in the nasal mask device, collecting biohazardous waste generated during the procedure in a plurality of pouches formed in the nasal mask device, and sealing each access port 106 once the surgical tool inserted through it is removed.

The nasal region of the patient does not include the mouth of the patient. This enables intubation of the patient during the procedure. Hermetic sealing of the nasal region ensures that aerosolised particles released by the patient during the procedure are restricted within the nasal mask device and is not permitted to escape outside. This prevents disease transmission to the clinician or any other patient from such particles released by the patient during the procedure.

The nasal mask device comprises a plurality of tool passing channels 104. The plurality of tool passing channels 104 run longitudinally and are angled inwardly towards each other. Each tool passing channel has a bottom end 104a and a top end 104b. The top end 104b opens to a nostril 107 of the patient and the bottom end 104a has formed on it an access port 106 to enable introduction of surgical tools. Only one surgical tool can be introduced through an access port 106 at a time. However, the plurality of access ports 106 formed in the plurality of tool passing channels 104 ensures that multiple surgical tools can be introduced simultaneously during the procedure.

Further, each tool channel is capable of independent movement. This ensures that surgical tools can be introduced into the nostrils 107 of the patient at various angles during the procedure. As per said preferred embodiment, the number of tool passing channels is 4, and diameter of a surgical tool that can be inserted/used is six millimetres. Further, each access port 106 has formed adjacent to it at least one pouch. Each pouch is formed inside the tool passing channel 104 and opens to the top end 104b of the tool passing channel 104, i.e. to the nostrils of the patient. The plurality of pouches formed in the plurality of access ports 106 enables collection of biohazardous waste generated during the procedure, which in turn ensures that such potential disease-causing material is confined to the nasal mask device.

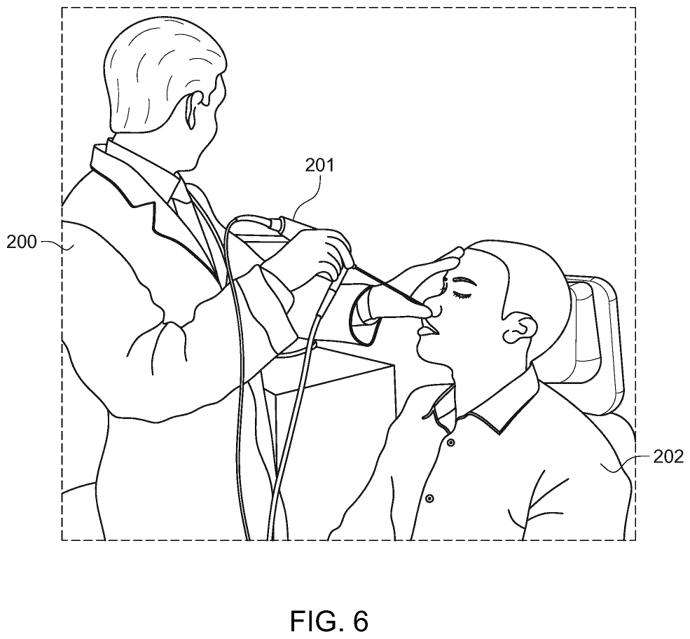

FIG. 5 illustrates a nasal mask device according to another embodiment of the present invention comprises a sealing means 101 and four access ports 106. Internal pouches 108 for the collection of materials can be incorporated into the flexible membrane for the collection of material by products of the surgical procedure. The operation of nasal mask device of FIG. 5 is the same as FIGS. 1 to 4, except the use of four access ports and channels allows more flexible operation.

FIG. 6 illustrates an example of a clinician 200 performing a minor nasal procedure in a non-surgical setting using a typical ENT tool device 201 for examining the inner nasal region of a patient 202. A problem with this approach is that the clinician can be infected by a patient carrying a disease by simply coughing or sneezing by the patient.

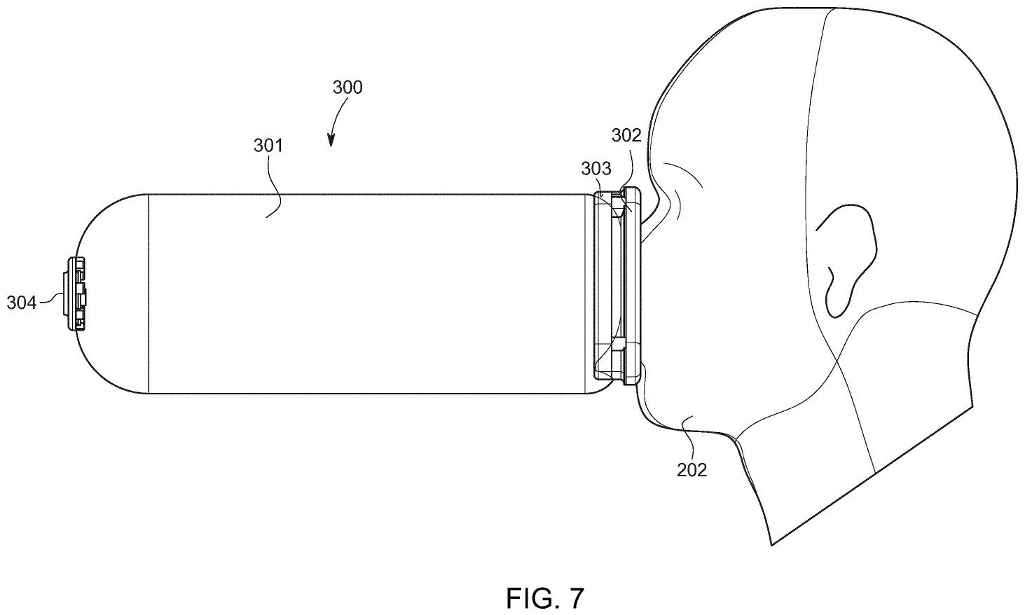

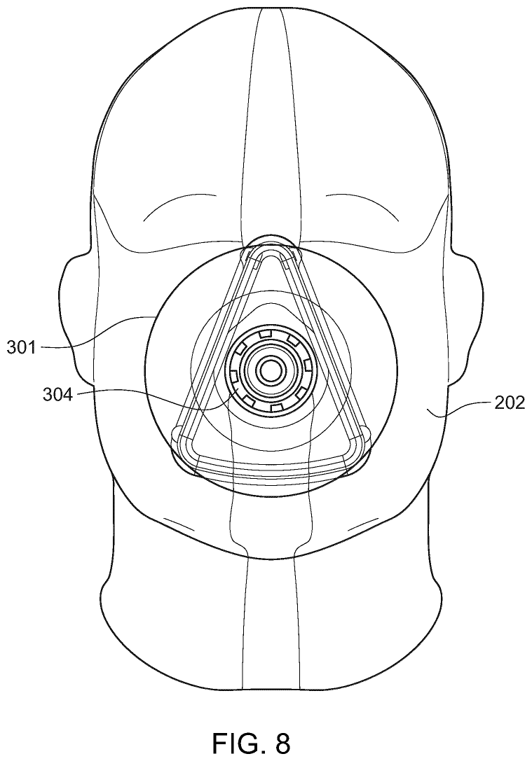

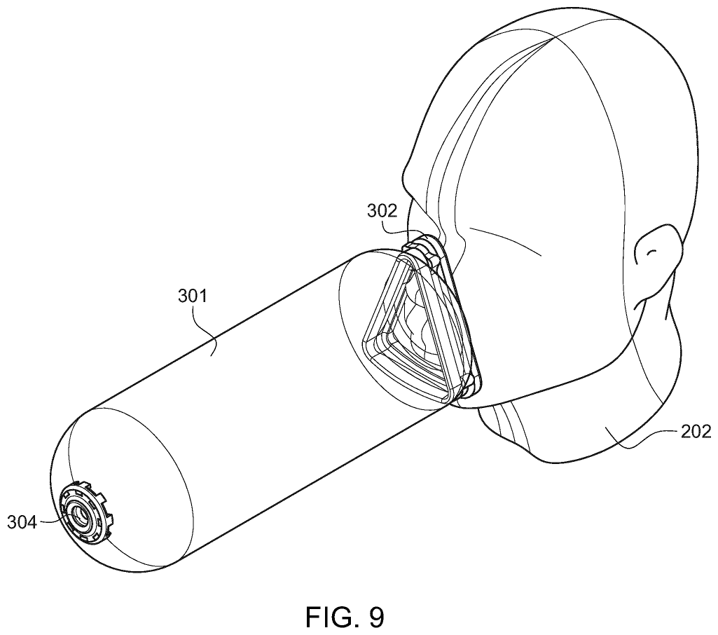

FIGS. 7, 8 and 9 illustrates another embodiment of the invention where a protective nasal mask device 300 is shown that works using a similar operation to the nasal mask device of FIGS. 1 to 5. In this embodiment an elongated flexible chamber 301 is provided and adapted at one end with a flexible sealing means 302 to seal the device to the nasal region of a user or patient 202. The elongated flexible chamber 301 defines a large volume to easily accommodate a typical ENT device (for example as shown in FIG. 6). The elongated flexible chamber 301 engages at one end a flexible membrane 303 attached to the device body opposite of the sealing means which covers a patient 202 nasal and/or mouth area. In the embodiment shown the nasal and mouth area are covered. At the opposite end an access port 304 is provided and adapted for insertion of an ENT tool, for example tool 201. The access port 304 is adapted to seal itself once a surgical tool 201 is removed from it. This ensures non permeation of any disease-causing pathogens from the nasal mask device and operates in a similar manner to the access ports described with respect to the protective nasal mask device of FIGS. 1 to 5.

A further access port (not shown) is positioned near the nasal or mouth area of the patient 202 to enable normal breathing by the patient. The access port can be configured to cooperate with a filter. For example, a simple embodiment is to take a standard CPAP filter and fit over the access port. The filter can also be integrated into the access port during manufacture. The function of the filter is to remove any pathogens from the patient when exhaling air prevents exposure of biohazardous waste to the clinician or surgeon.

Preferably the elongated flexible chamber 301 is made from a flexible transparent material that can be adjusted such that the chamber can collapse in on itself for easy storage or selected to be a particular size by the clinician depending on the dimension of the ENT tool 202 to be inserted during use.

The method described above thus enables performance of ENT procedures without exposing the clinician to disease causing pathogens. The nasal mask device as per the present invention is for single use and shall be carefully disposed after the procedure.

In the specification, the terms “comprise, comprises, comprised and comprising” or any variation thereof and the terms include, includes, included and including” or any variation thereof are considered to be totally interchangeable and they should all be afforded the widest possible interpretation and vice versa.

Although the present invention has been described with reference to specific embodiments, this description is not meant to be construed in a limiting sense. Various modifications of the disclosed embodiments, as well as alternate embodiments of the subject matter, will become apparent to persons skilled in the art upon reference to the description of the subject matter.

It is therefore contemplated that such modifications can be made without departing from the spirit or scope of the present invention as defined.

1. A protective nasal mask device comprising:
a sealing means adapted to seal the device to the nasal region of a user, the sealing means having a bottom edge and a top edge disposed opposite to the bottom edge;
a device body coupled to the top edge of the sealing means;
a flexible membrane attached to the device body opposite to the sealing means and comprising a plurality of longitudinally running tool passing channels mounted on the flexible member and angled inwardly towards each other; and
each tool passing channel having a bottom end with an access port formed on it and a top end kept open, wherein the flexible member enables independent movement of each tool passing channel and wherein each access port is adapted to seal itself when a surgical tool inserted through it is removed.

2. The protective nasal mask device as claimed in claim 1, wherein the bottom edge of the sealing means is adapted to hermetically seal against the nasal region of the user.

3. The protective nasal mask device as claimed in any of the preceding claims, wherein the device body has mounted on it a plurality of fixing points capable of securing the device to the nasal region of the user.

4. The protective nasal mask device as claimed in any of the preceding claims, wherein the plurality of tool passing channels have a hollow flexible interior.

5. The protective nasal mask device as claimed in any of the preceding claims, wherein the top end of each tool passing channel opens to a nostril of the user.

6. The protective nasal mask device as claimed in any of the preceding claims, wherein the number of tool passing channels is four.

7. The protective nasal mask device as claimed in any of the preceding claims wherein the device body is comprised of transparent plastic.

8. The protective nasal mask device as claimed in any of the preceding claims, wherein the flexible member is comprised of elastomeric material.

9. The protective nasal mask device as claimed in any of the preceding claims, wherein the nasal region does covers the mouth and nasal region of the user.

10. The protective nasal mask device as claimed in any or the preceding claims wherein at least one of the tool passing channels is adapted to provide an air passageway to enable the user to breathe.

11. The protective nasal mask as claimed in any of the preceding claims, wherein at least one access port comprises at least one pouch formed adjacent to it and inside the tool passing channel such that the pouch opens towards the top end of the tool passing channel.

12. The protective nasal mask as claimed in claim 11 wherein the pouch is designed to securely store hazardous material from a patient during a procedure.

13. The protective nasal mask as claimed in claim 11 wherein at least one pouch is adapted as a store area for storage of materials required during a procedure.

14. A method for performing an Ears, Nose and Throat (ENT) Procedure, the method comprising the steps of:
a) sealing the nasal region of a patient using a nasal mask device, the nasal device comprising a plurality of longitudinally running tool passing channels angled inwardly towards each other, each tool passing channel having a bottom end with an access port formed on it and a top end opening to a nostril of the patient, each access port having at least one pouch formed adjacent to it and inside the tool passing channel such that each pouch opens towards the top end of the tool passing channel, wherein each of the plurality of tool passing channels is capable of independent movement;
b) introducing at least one surgical tool through the plurality of access ports, wherein at a time only one surgical tool can be introduced through each access port;
c) collecting biohazardous waste generated during the procedure in one or more pouches; and
d) sealing each access port when the surgical tool is removed.

15. The method as claimed in claim 14, wherein the number of tool passing channels is four.

16. The method as claimed in claim 14 or 15, wherein the nasal region covers the mouth and nasal region of the user.

17. A protective nasal mask device comprising:
a sealing means adapted to seal the device to the nasal region of a user, the sealing means having a bottom edge and a top edge disposed opposite to the bottom edge;
a device body coupled to the top edge of the sealing means, the device body having a flexible member integrated to its bottom edge; and
at least one longitudinally running tool passing channel mounted on the flexible member and angled inwardly towards each other, the tool passing channel having a bottom end with an access port formed on it and a top end kept open, wherein the flexible member enables independent movement of the tool passing channel and wherein the access port is adapted to seal itself when a surgical tool inserted through it is removed.

18. The protective nasal mask device as claimed in claim 17 wherein the device comprises at least one pouch formed adjacent to it and inside the tool passing channel such that the pouch opens towards the top end of the tool passing channel.

19. The protective nasal mask device as claimed in claim 17 or 18 wherein the device and tool passing channel comprises an elongated flexible chamber and coupled at one end with a flexible sealing means.

20. The protective nasal mask as claimed in claim 19 wherein the access port is positioned at the opposite end of the elongated flexible chamber.

21. The protective nasal mask as claimed in claim 19 or 20 wherein a second access port is positioned near the flexible sealing means and configured to provide an air passageway to enable a patient to breathe.

22. The protective nasal mask as claimed in any of claim 19, 20 or 21 wherein the elongated flexible chamber comprises a transparent material.
